(19) United States
(12) Patent Application Publication
(10) Pub. No.: US 2004/0131572 A1
(43) Pub. Date: Jul. 8, 2004

(54) Composition for treating keratinous materials comprising a cationic poly(alkyl) vinyllactam polymer and a protecting or conditioning agent

(76) Inventors: Francois Cottard, Perret (FR); Roland De La Mettrie, Vesinet (FR)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/470,195

(22) PCT Filed: Jan. 22, 2002
(86) PCT No.: PCT/FR02/00251

(30) Foreign Application Priority Data
Jan. 26, 2001 (FR) 01/01108

(51) Int. Cl. C08K 5/34; A61K 7/06; A61K 7/11

(52) U.S. Cl. 424/70.16; 524/91

(57) ABSTRACT

The invention relates to a composition for treating keratin materials, in particular the hair, comprising, in a physiologically and in particular a cosmetically acceptable medium, at least one protecting or conditioning agent, and also at least one cationic poly(alkyl)vinyllactam polymer.

These combinations make it possible to improve the deposition of the agent for protecting or conditioning the keratin materials and also the cosmetic properties.

The invention relates to a composition for treating keratin materials, in particular human keratin fibers such as the hair, comprising, in a physiologically and in particular a cosmetically acceptable medium, at least one protecting or conditioning agent, and also at least one cationic poly(alkyl)vinyllactam polymer.

The deposition of the agent for protecting or conditioning keratin fibers and the cosmetic properties may be improved with these combinations.

It is well known that the hair is sensitized or embrittled to varying degrees by the action of atmospheric agents and especially light, water and humidity, and also by the repeated action of various hair treatments such as washing, permanent-waving, straightening, dyeing and bleaching. Many publications disclose that natural light destroys certain amino acids of the hair. These attacks impair the hair fibers and reduce their mechanical properties, for instance the tensile strength, the breaking load and the elasticity, or their resistance to swelling in an aqueous medium. The hair then becomes dull, coarse and brittle. In contrast with the skin, the color of the hair becomes lighter.

It is also known that light and washing agents, especially, have a tendency to attack the natural color of the hair and also the artificial color of dyed hair. The color of the hair gradually fades or turns to unattractive or undesirable shades.

Substances for protecting the hair against the degradation caused by atmospheric attacking factors, such as light, heat and treatments, have been sought for many years in the cosmetics industry. In particular, products that protect the color of naturally colored or artificially dyed keratin fibers and that preserve or reinforce the intrinsic mechanical properties of keratin fibers (the tensile strength, the breaking load and the elasticity, or their resistance to swelling in an aqueous medium) are particularly sought.

It has already been proposed to use certain substances capable of screening out light radiation, for instance 2-hydroxy-4-methoxybenzophenone-5-sulfonic acid or its salts (FR-A-2 627 085), 4-(2-oxo-3-bornylidenemethyl)benzenesulfonic acid or its salts (EP-A-329 032) or lactoferrin (FR-A-2 673 839), to combat this degradation of hair keratin.

However, these screening agents, when they are effective, are only effective in large concentrations. At these concentrations though, hair treated with these screening agents has a coarse, heavy feel. Furthermore, it is extremely difficult to disentangle.

It has already been recommended, in compositions for washing or caring for keratin materials such as the hair, to use conditioners, especially cationic polymers or silicones, to facilitate the disentangling of the hair and to give it softness and suppleness. However, the cosmetic advantages mentioned above are unfortunately also accompanied, on dried hair, by certain cosmetic effects considered undesirable, i.e. lankness of the hairstyle (lack of lightness of the hair) and lack of smoothness (hair that is not uniform from the root to the end).

In addition, the use of cationic polymers with this aim presents various drawbacks. On account of their high affinity for the hair, some of these polymers become deposited to a substantial amount during repeated use, and lead to undesirable effects such as an unpleasant, heavy feel, stiffening of the hair and adhesion between the fibers that affects the styling. These drawbacks are accentuated in the case of fine hair that lacks liveliness and body.

It is well known that hair that has been sensitized (i.e. damaged and/or embrittled) to varying degrees due to the action of atmospheric agents or due to the action of mechanical or chemical treatments, such as dyeing, bleaching and/or permanent-waving operations, is often difficult to disentangle and to style, and lacks softness.

In summary, it is found that the current cosmetic compositions containing protecting or conditioning agents are not entirely satisfactory.

The Applicant has now discovered that the combination of a particular cationic poly(alkyl)vinyllactam polymer with protecting or conditioning agents allows these drawbacks to be overcome.

Thus, after considerable research conducted in this matter, it has now been found by the Applicant that by introducing a particular polymer into compositions, in particular hair compositions based on agents for protecting or conditioning keratin materials, it is possible to increase the deposition of the agent for protecting or conditioning keratin materials and thereby to increase the protection or conditioning.

Without wishing to limit the present invention to any theory, it is believed that, during rinsing, there are particular interactions and/or affinities between the agent for protecting or conditioning keratin materials, the poly(alkyl)vinyllactam polymers in accordance with the invention and the hair, which promote a uniform, substantial and long-lasting deposition of said agents for protecting or conditioning keratin materials and poly(alkyl)vinyllactam polymers at the surface of said hair, this qualitative and quantitative deposition probably being one of the causes of the observed improvement in the final properties, in particular the ease of styling, the hold, the liveliness and the body of the treated hair.

All these discoveries form the basis of the present invention.

Thus, according to the present invention, novel cosmetic compositions are now proposed, comprising, in a physiologically and in particular a cosmetically acceptable medium, at least one agent for protecting or conditioning keratin materials and at least one cationic poly(alkyl)vinyllactam polymer comprising:

a) at least one monomer of vinyllactam or (C₁-C₅)alkylvinyllactam type;

b) at least one monomer chosen from those of structures (I) and (II):

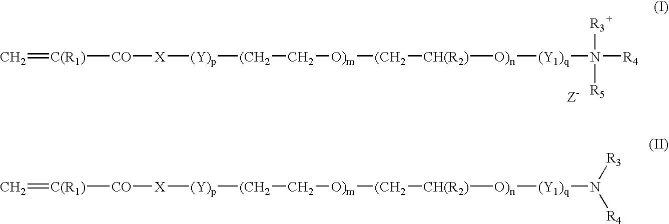

in which:

X denotes an oxygen atom or a radical NR₆,

R₁ and R₆ denote, independently of each other, a hydrogen atom or a linear or branched C₁-C₅ alkyl radical,

R₂ denotes a linear or branched C₁-C₄ alkyl radical,

R₃, R₄ and R₅ denote, independently of each other, a hydrogen atom, a linear or branched C₁-C₃₀ alkyl radical or a radical of formula (III):

—(Y₂)ᵣ— (CH₂—CH(R₇)—O)ₓ—R₈ (III)

Y, Y₁ and Y₂ denote, independently of each other, a linear or branched C₂-C₁₆ alkylene radical,

R₇ denotes hydrogen, a linear or branched C₁-C₄ alkyl radical or a linear or branched C₁-C₄ hydroxyalkyl radical,

R₈ denotes a hydrogen atom or a linear or branched C₁-C₃₀ alkyl radical,

p, q and r denote, independently of each other, either the value 0 or the value 1,

m and n denote, independently of each other, an integer ranging from 0 to 100,

x denotes an integer ranging from 1 to 100,

Z denotes an organic or mineral acid anion,

with the proviso that:

at least one of the substituents R₃, R₄, R₅ or R₈ denotes a linear or branched C₉-C₃₀ alkyl radical,

if m or n is other than 0, then q is equal to 1,

if m or n are equal to 0, then either p or q is equal to 0.

Another subject of the invention relates to the use of at least one cationic poly(alkyl)vinyllactam polymer in or for the manufacture of a cosmetic composition comprising an agent for protecting or conditioning keratin materials.

A subject of the invention is also the use of at least one cationic poly(alkyl)vinyllactam polymer in a cosmetic composition comprising a protecting agent for keratin materials, to increase the efficacy of this agent for protecting or conditioning keratin materials.

A subject of the present invention is also the use of at least one cationic poly(alkyl)vinyllactam polymer in a composition comprising an agent for protecting or conditioning keratin materials, to improve the deposition and/or fixing of said protecting agent to the keratin materials.

The various subjects of the invention will now be presented in detail. All the meanings and definitions of the compounds used in the present invention given hereinbelow are valid for all the subjects of the invention.

Without wishing to be bound by any theory, it is believed that the advantages afforded by the polymers of the invention are in relation with a behavior of thickening polymers of associative type.

Associative polymers are polymers whose molecules are capable, in the formulation medium, of associating with each other or with molecules of other compounds.

Their chemical structure generally comprises at least one hydrophilic region and at least one hydrophobic region, the hydrophobic region(s) comprising at least one fatty chain.

The cationic poly(alkyl)vinyllactam polymers according to the invention comprise:

a) at least one monomer of vinyllactam or (C₁-Cs)alkylvinyllactam type;

b) at least one monomer chosen from those of structures (I) and (II):

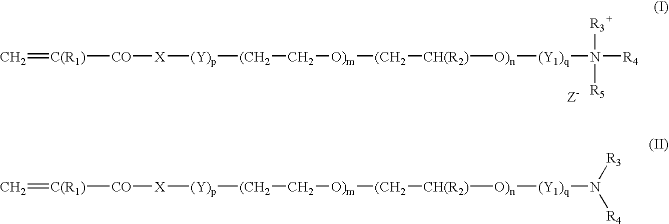

in which:

X denotes an oxygen atom or a radical NR₆,

R₁ and R₆ denote, independently of each other, a hydrogen atom or a linear or branched C₁-C₅ alkyl radical,

R₂ denotes a linear or branched C₁-C₄ alkyl radical,

R₃, R₄ and R₅ denote, independently of each other, a hydrogen atom, a linear or branched C₁-C₃₀ alkyl radical or a radical of formula (III):

—(Y₂)ᵣ—(CH₂—CH(R₇)—O)ₓ—R₈ (III)

Y, Y₁ and Y₂ denote, independently of each other, a linear or branched C₂-C₁₆ alkylene radical,

R₇ denotes hydrogen, a linear or branched C₁-C₄ alkyl radical or a linear or branched C₁-C₄ hydroxyalkyl radical,

R₈ denotes hydrogen or a linear or branched C₁-C₃₀ alkyl radical,

p, q and r denote, independently of each other, either the value 0 or the value 1,

m and n denote, independently of each other, an integer ranging from 0 to 100,

x denotes an integer ranging from 1 to 100,

Z denotes an organic or mineral acid anion,

with the proviso that:

at least one of the substituents R₃, R₄, R₅ or R₈ denotes a linear or branched C₉-C₃₀ alkyl radical,

if m or n is other than 0, then q is equal to 1,

if m or n are equal to 0, then either p or q is equal to 0.

The polymers according to the invention may be crosslinked or non-crosslinked and may also be block polymers.

Preferably, the counterion Z⁻ of the monomers of formula (I) is chosen from halide ions, phosphate ions, methosulfate ions and tosylate ions.

Preferably, R₃, R₄ and R₅ in formula(e) (I) and/or (II) denote, independently of each other, a hydrogen atom or a linear or branched C₁-C₃₀ alkyl radical.

More preferably, the monomer b) is a monomer of formula (I) and, even more preferably, m and n are equal to 0.

The vinyllactam or alkylvinyllactam monomer is preferably a compound of structure (IV):

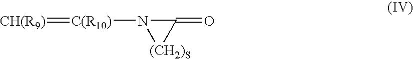

in which:

s denotes an integer ranging from 3 to 6,

R₉ denotes a hydrogen atom or a C₁-C₅ alkyl radical,

R₁₀ denotes a hydrogen atom or a C₁-C₅ alkyl radical, with the proviso that at least one of the radicals R₉ and

R₁₀ denotes a hydrogen atom.

Even more preferably, the monomer (IV) is vinylpyrrolidone.

The polymers according to the invention may also contain one or more additional monomers, preferably cationic or nonionic monomers.

As compounds that are more particularly preferred, mention will be made of terpolymers comprising at least:

a) one monomer of formula (IV),

b) one monomer of formula (I) in which p=1, q=0, R₃ and R₄ denote a hydrogen atom or a C₁-C₅ alkyl radical and R₅ denotes a C₉-C₂₄ alkyl radical, and m and n are zero, and

c) one monomer of formula (II) in which R₃ and R₄ denote a hydrogen atom or a C₁-C₅ alkyl radical, and m and n are zero.

Even more preferably, poly(alkyl)vinyllactam terpolymers comprising, by weight, 40% to 95% of monomer (a), 25% to 50% of monomer (b) and 0.1% to 55% of monomer (c) will be used.

Such polymers are described in patent application WO 00/68282, the content of which forms an integral part of the invention.

As cationic poly(alkyl)vinyllactam polymers according to the invention, vinylpyrrolidone/dimethylaminopropylmethacrylamide/dodecyldimethylmethacrylamidopropylammonium tosylate terpolymers, vinylpyrrolidone/dimethylaminopropylmethacrylamide/cocoyldimethylmethacrylamidopropylammonium tosylate terpolymers, or vinylpyrrolidone/dimethylaminopropylmethacrylamide/lauryldimethylmethacrylamidopropylammonium tosylate or chloride terpolymers are used in particular.

The weight-average molecular mass of the cationic polymers of the invention is preferably between 500 and 20 000 000. It is more particularly between 200 000 and 2 000 000 and even more preferably between 400 000 and 800 000.

The cationic poly(alkyl)vinyllactam polymers are preferably used in an amount that can range from about 0.01% to 10% by weight relative to the total weight of the composition for treating keratin materials. This amount more preferably ranges from about 0.1% to 5% by weight.

The agents for protecting keratin materials may be any active agent that is useful for preventing or limiting degradation caused by physical or chemical attack.

Thus, the agent for protecting keratin materials may be chosen from water-soluble, liposoluble or water-insoluble organic UV-screening agents, free-radical scavengers, antioxidants, vitamins and provitamins.

The organic UV-screening agents (systems for screening out UV radiation) are chosen especially from water-soluble or liposoluble, silicone or nonsilicone screening agents and mineral oxide nanoparticles which have optionally been surface-treated to make them hydrophilic or hydrophobic.

The water-soluble UV-screening agents may be chosen, for example, from para-aminobenzoic acid and its salts, anthranilic acid and its salts, salicylic acid and its salts, p-hydroxycinnamic acid and its salts, sulfonic derivatives of benz-x-azoles (benzothiazoles, benzimidazoles and benzoxazoles) and salts thereof, sulfonic derivatives of benzophenone and salts thereof, sulfonic derivatives of benzylidenecamphor and salts thereof, benzylidenecamphor derivatives substituted with a quaternary amine and salts thereof, phthalylidene-camphorsulfonic acid derivatives and salts thereof, and sulfonic derivatives of benzotriazole, and mixtures thereof.

It is also possible to use hydrophilic polymers having, in addition and on account of their chemical nature, UV-photoprotective properties. Mention may be made of polymers comprising benzylidenecamphor and/or benzotriazole groups, substituted with sulfonic or quaternary ammonium groups.

As liposoluble (or lipophilic) organic UV-screening agents that are suitable for use in the present invention, mention may be made especially of: p-aminobenzoic acid derivatives, such as p-aminobenzoic acid esters or amides; salicylic acid derivatives such as the esters; benzophenone derivatives; dibenzoylmethane derivatives; diphenylacrylate derivatives; benzofuran derivatives; polymeric UV-screening agents containing one or more organosilicon residues; cinnamic acid esters; camphor derivatives; trianilino-s-triazine derivatives; urocanic acid ethyl ester; benzotriazoles; hydroxyphenyltriazine derivatives; bis-resorcinol-dialkylaminotriazines; and mixtures thereof.

The liposoluble (or lipophilic) UV-screening agent according to the invention is preferably chosen from: octyl salicylate; 4-tert-butyl-4′-methoxydibenzoylmethane (Parsol 1789 from Givaudan); octocrylene; 2-ethylhexyl 4-methoxycinnamate (Parsol MCX) and the compound of formula (V) below, or 2-(2H-benzotriazol-2-yl)-4-methyl-6-[2-methyl-3-[1,3,3,3-tetramethyl-1-[(trimethylsilyl)oxy]disiloxanyl]propynyl]phenol, described in patent application EP-A-0 392 883:

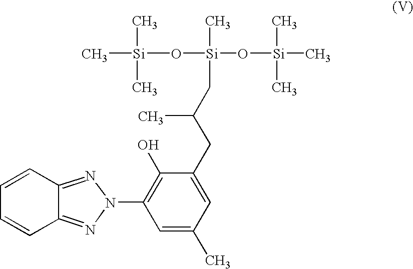

Other UV-screening agents that are particularly preferred according to the invention are benzophenone derivatives such as Uvinul MS 40 (2-hydroxy-4-methoxybenzophenone-5-sulfonic acid) and Uvinul M40 (2-hydroxy-4-methoxybenzophenone) sold by BASF, benzalmalonate derivatives such as Parsol SLX (polydimethyl/methyl (3-(4-(2,2-bis-ethoxycarbonylvinyl)phenoxy)propenyl)siloxane) sold by Givaudan-Rouré, benzylidenecamphor derivatives such as Mexoryl SX (β,β′-camphorsulfonic [1,4-divinylbenzene] acid) manufactured by the company Chimex, and benzimidazole derivatives such as Eusolex 232 (2-phenylbenzimidazole-5-sulfonic acid) sold by Merck.

The mineral oxides may be chosen from titanium oxides, zinc oxides and cerium oxides.

The antioxidants and/or free-radical scavengers are chosen especially from phenols such as BHA (tert-butyl-4-hydroxyanisole), BHT (2,6-di-tert-butyl-p-cresol), TBHQ (tert-butylhydroquinone), polyphenols such as proanthocyanidol oligomers and flavonoids, hindered amines known under the generic term HALS (Hindered Amine Light Stabilizer) such as tetraminopiperidine, erythorbic acid, polyamines such as spermine, cysteines, glutathione, superoxide dismutase and lactoferrin.

The vitamins are chosen especially from ascorbic acid, vitamin E, vitamin E acetate, B vitamins such as vitamins B3 and B5, vitamin PP, and vitamin A and its derivatives.

The provitamins are chosen especially from panthenol and retinol.

The sequestering agents are chosen especially from the Dequest products such as diethylenetriamine-pentamethylenephosphonic acid and diethylenetriamine-tetramethylenephosphonic acid and salts thereof, EDTA (ethylenediaminetetraacetic acid) and its salts, especially the sodium or potassium salts.

According to the invention, the protecting agent(s) for keratin materials may represent from 0.001% to 20% by weight, preferably from 0.01% to 10% by weight and more particularly from 0.1% to 5% by weight, relative to the total weight of the final composition.

In the context of the present patent application, the term “conditioner” means any agent whose function is to improve the cosmetic properties of the hair, in particular the softness, disentangling, feel and static electricity.

The conditioners may be in liquid, semi-solid or solid form such as, for example, oils, waxes or gums.

According to the invention, the conditioners may be chosen from synthetic oils such as polyolefins, mineral oils, plant oils, fluoro oils or perfluoro oils, natural or synthetic waxes, silicones, cationic polymers, compounds of ceramide type, cationic surfactants, fatty amines, fatty acids and derivatives thereof, fatty alcohols and derivatives thereof, and also mixtures of these various compounds.

The conditioners that are preferred according to the invention are cationic polymers and silicones.

The synthetic oils are especially polyolefins, in particular poly-α-olefins and more particularly:

of hydrogenated or nonhydrogenated polybutene type, and preferably hydrogenated or nonhydrogenated polyisobutene.

Isobutylene oligomers with a molecular weight of less than 1000 and mixtures thereof with polyisobutylenes with a molecular weight of greater than 1000, and preferably between 1000 and 15 000, are preferably used.

As examples of poly-α-olefins that can be used in the context of the present invention, mention may be made more particularly of the products sold under the name Permethyl 99 A, 101 A, 102 A, 104 A (n=16) and 106 A (n=38) by the company Presperse Inc., or alternatively the products sold under the name Arlamol HD (n=3) by the company ICI (n denoting the degree of polymerization),

of hydrogenated or nonhydrogenated polydecene type.

Such products are sold, for example, under the names Ethylflo by the company Ethyl Corp. and Arlamol PAO by the company ICI.

The mineral oils that may be used in the compositions of the invention are preferably chosen from the group formed by:

hydrocarbons, such as hexadecane and liquid paraffin.

The animal or plant oils are preferably chosen from the group formed by sunflower oil, corn oil, soybean oil, avocado oil, jojoba oil, marrow oil, grapeseed oil, sesame oil, hazelnut oil, fish oils, glyceryl tricaprocaprylate, or plant or animal oils of formula R₉COOR₁₀ in which R₉ represents a higher fatty acid residue containing from 7 to 29 carbon atoms and R₁₀ represents a linear or branched hydrocarbon-based chain containing from 3 to 30 carbon atoms, in particular alkyl or alkenyl, for example purcellin oil or liquid jojoba wax.

It is also possible to use natural or synthetic essential oils such as, for example, eucalyptus oil, lavandin oil, lavender oil, vetiver oil, Litsea cubeba oil, lemon oil, sandalwood oil, rosemary oil, camomile oil, savory oil, nutmeg oil, cinnamon oil, hyssop oil, caraway oil, orange oil, geraniol oil, cade oil and bergamot oil.

The waxes are natural (animal or plant) or synthetic substances that are solid at room temperature (20°-25° C.). They are insoluble in water, soluble in oils and are capable of forming a water-repellent film.

For the definition of waxes, mention may be made, for example, of P. D. Dorgan, Drug and Cosmetic Industry, December 1983, pp. 30-33.

The wax(es) is(are) chosen in particular from carnauba wax, candelilla wax, esparto wax, paraffin wax, ozokerite, plant waxes such as olive tree wax, rice wax, hydrogenated jojoba wax or the absolute waxes of flowers such as the essential wax of blackcurrant flower sold by the company Bertin (France), animal waxes such as beeswaxes, or modified beeswaxes (cerabellina); other waxes or waxy starting materials which can be used according to the invention are, in particular, marine waxes such as the product sold by the company Sophim under the reference M82, and polyethylene waxes or polyolefins in general.

The cationic polymers (other than the poly(alkyl)vinyllactams according to the invention) that may be used in accordance with the present invention may be chosen from all those already known per se as improving the cosmetic properties of hair treated with detergent compositions, i.e. especially those described in patent application EP-A-337 354 and in French patent applications FR-A-2 270 846, 2 383 660, 2 598 611, 2 470 596 and 2 519 863.

Even more generally, for the purpose of the present invention, the term “cationic polymer” denotes any polymer containing cationic groups and/or groups that may be ionized into cationic groups.

The cationic polymers that are preferred are chosen from those containing units comprising primary, secondary, tertiary and/or quaternary amine groups that may either form part of the main polymer chain or be borne by a side substituent directly attached thereto.

The cationic polymers used generally have a number-average molecular mass of between 500 and 5×10⁶ approximately and preferably between 103 and 3×10⁶ approximately.

Among the cationic polymers that may be mentioned more particularly are polymers of the polyamine, polyamino amide and polyquaternary ammonium type. These are known products.

The polymers of the polyamine, polyamino amide and polyquaternary ammonium type that may be used in accordance with the present invention, and that may especially be mentioned, are those described in French patents No.s 2 505 348 and 2 542 997. Among these polymers, mention may be made of:

(1) homopolymers or copolymers derived from acrylic or methacrylic esters or amides and comprising at least one of the units of the following formulae:

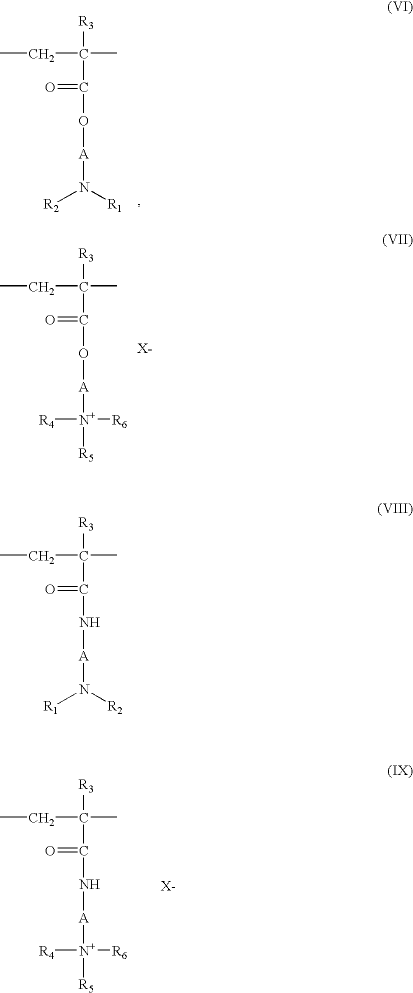

in which:

R₁ and R₂, which may be identical or different, represent hydrogen or an alkyl group containing from 1 to 6 carbon atoms, and preferably methyl or ethyl;

R₃, which may be identical or different, denote a hydrogen atom or a CH₃ radical;

A, which may be identical or different, represent a linear or branched alkyl group of 1 to 6 carbon atoms, preferably 2 or 3 carbon atoms, or a hydroxyalkyl group of 1 to 4 carbon atoms;

R₄, R₅ and R₆, which may be identical or different, represent an alkyl group containing from 1 to 18 carbon atoms or a benzyl radical and preferably an alkyl group containing from 1 to 6 carbon atoms;

X denotes an anion derived from a mineral or organic acid, such as a methosulfate anion or a halide such as chloride or bromide.

The copolymers of family (1) can also contain one or more units derived from comonomers which may be chosen from the family of acrylamides, methacrylamides, diacetone acrylamides, acrylamides and methacrylamides substituted on the nitrogen with lower (C₁-C₄) alkyls, acrylic or methacrylic acids or esters thereof, vinyllactams such as vinylpyrrolidone or vinylcaprolactam, and vinyl esters.

Thus, among these copolymers of family (1), mention may be made of:

copolymers of acrylamide and of dimethylaminoethyl methacrylate quaternized with dimethyl sulfate or with a dimethyl halide, such as the product sold under the name Hercofloc by the company Hercules,

the copolymers of acrylamide and of methacryloyloxyethyltrimethylammonium chloride described, for example, in patent application EP-A-080 976 and sold under the name Bina Quat P 100 by the company Ciba Geigy,

the copolymer of acrylamide and of methacryloyloxyethyltrimethylammonium methosulfate sold under the name Reten by the company Hercules,

quaternized or nonquaternized vinylpyrrolidone/dialkylaminoalkyl acrylate or methacrylate copolymers, such as the products sold under the name Gafquat by the company ISP, such as, for example, Gafquat 734 or Gafquat 755, or alternatively the products known as Copolymer 845, 958 and 937. These polymers are described in detail in French patents 2 077 143 and 2 393 573,

dimethylaminoethyl methacrylate/vinylcaprolactam/vinylpyrrolidone terpolymers, such as the product sold under the name Gaffix VC 713 by the company ISP,

vinylpyrrolidone/methacrylamidopropyldimethylamine copolymers sold in particular under the name Styleze CC 10 by ISP, and

quaternized vinylpyrrolidone/dimethylaminopropylmethacrylamide copolymers, such as the product sold under the name Gafquat HS 100 by the company ISP.

(2) The cellulose ether derivatives containing quaternary ammonium groups, described in French patent 1 492 597, and in particular polymers sold under the name “JR” (JR 400, JR 125 and JR 30M) or “LR” (LR 400 or LR 30M) by the company Union Carbide Corporation. These polymers are also defined in the CTFA dictionary as quaternary ammoniums of hydroxyethylcellulose that have reacted with an epoxide substituted with a trimethylammonium group.

(3) Cationic cellulose derivatives such as cellulose copolymers or cellulose derivatives grafted with a water-soluble quaternary ammonium monomer, and described in particular in U.S. Pat. No. 4,131,576, such as hydroxyalkylcelluloses, for instance hydroxymethyl-, hydroxyethyl- or hydroxypropylcelluloses grafted, in particular, with a methacryloylethyltrimethylammonium, methacrylamidopropyltrimethylammonium or dimethyldiallylammonium salt.

The commercial products corresponding to this definition are more particularly the products sold under the names Celquat L 200 and Celquat H 100 by the company National Starch.

(4) The cationic polysaccharides described more particularly in U.S. Pat. Nos. 3,589,578 and 4,031,307, such as guar gums containing cationic trialkylammonium groups. Guar gums modified with a salt (e.g. chloride) of 2,3-epoxypropyltrimethylammonium are used, for example.

Such products are sold in particular under the trade names Jaguar C13 S, Jaguar C 15, Jaguar C 17 and Jaguar C162 by the company Meyhall.

(5) Polymers consisting of piperazinyl units and of divalent alkylene or hydroxyalkylene radicals containing straight or branched chains, optionally interrupted by oxygen, sulfur or nitrogen atoms or by aromatic or heterocyclic rings, as well as the oxidation and/or quaternization products of these polymers. Such polymers are described, in particular, in French patents 2 162 025 and 2 280 361.

(6) Water-soluble polyamino amides prepared in particular by polycondensation of an acidic compound with a polyamine; these polyamino amides can be crosslinked with an epihalohydrin, a diepoxide, a dianhydride, an unsaturated dianhydride, a bis-unsaturated derivative, a bis-halohydrin, a bis-azetidinium, a bis-haloacyldiamine, a bis-alkyl halide or alternatively with an oligomer resulting from the reaction of a difunctional compound which is reactive with a bis-halohydrin, a bis-azetidinium, a bis-haloacyldiamine, a bis-alkyl halide, an epihalohydrin, a diepoxide or a bis-unsaturated derivative; the crosslinking agent being used in proportions ranging from 0.025 to 0.35 mol per amine group of the polyamino amide; these polyamino amides can be alkylated or, if they contain one or more tertiary amine functions, they can be quaternized. Such polymers are described, in particular, in French patents 2 252 840 and 2 368 508.

(7) The polyamino amide derivatives resulting from the condensation of polyalkylene polyamines with polycarboxylic acids followed by alkylation with difunctional agents. Mention may be made, for example, of adipic acid/dialkylaminohydroxyalkyldialkylenetriamine polymers in which the alkyl radical contains from 1 to 4 carbon atoms and preferably denotes methyl, ethyl or propyl. Such polymers are described in particular in French patent 1 583 363.

Among these derivatives, mention may be made more particularly of the adipic acid/dimethylaminohydroxypropyl/diethylenetriamine polymers sold under the name Cartaretine F, F4 or F8 by the company Sandoz.

(8) The polymers obtained by reaction of a polyalkylene polyamine containing two primary amine groups and at least one secondary amine group with a dicarboxylic acid chosen from diglycolic acid and saturated aliphatic dicarboxylic acids having from 3 to 8 carbon atoms. The molar ratio between the polyalkylene polyamine and the dicarboxylic acid is between 0.8:1 and 1.4:1; the polyamino amide resulting therefrom is reacted with epichlorohydrin in a molar ratio of epichlorohydrin relative to the secondary amine group of the polyamino amide of between 0.5:1 and 1.8:1. Such polymers are described in particular in U.S. Pat. Nos. 3,227,615 and 2,961,347.

Polymers of this type are sold in particular under the name Hercosett 57 by the company Hercules Inc. or alternatively under the name PD 170 or Delsette 101 by the company Hercules in the case of the adipic acid/epoxypropyl/diethylenetriamine copolymer.

(9) Cyclopolymers of alkyldiallylamine or of dialkyldiallylammonium, such as the homopolymers or copolymers containing, as main constituent of the chain, units corresponding to formula (X) or (XI):

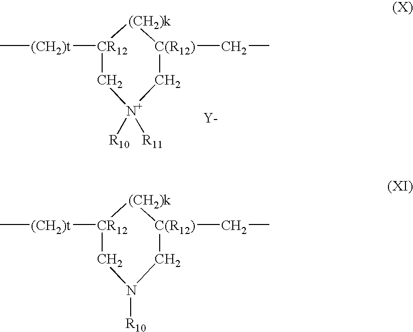

in which formulae k and t are equal to 0 or 1, the sum k+t being equal to 1; R₁₂ denotes a hydrogen atom or a methyl radical; R₁₀ and R₁₁, independently of each other, denote an alkyl group having from 1 to 22 carbon atoms, a hydroxyalkyl group in which the alkyl group preferably has 1 to 5 carbon atoms, a lower (C₁-C₄) amidoalkyl group, or R₁₀ and R₁₁ can denote, together with the nitrogen atom to which they are attached, heterocyclic groups such as piperidyl or morpholinyl; Y⁻ is an anion such as bromide, chloride, acetate, borate, citrate, tartrate, bisulfate, bisulfite, sulfate or phosphate. These polymers are described in particular in French patent 2 080 759 and in its Certificate of Addition 2 190 406.

R₁₀ and R₁₁, independently of each other, preferably denote an alkyl group containing from 1 to 4 carbon atoms.

Among the polymers defined above, mention may be made more particularly of the dimethyldiallylammonium chloride homopolymer sold under the name Merquat 100 by the company Calgon (and its homologs of low weight-average molecular mass) and the copolymers of diallyldimethylammonium chloride and of acrylamide, sold under the name Merquat 550.

(10) The quaternary diammonium polymer containing repeating units corresponding to the formula:

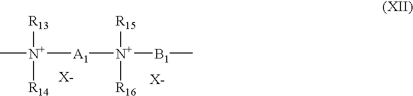

in which formula (VII):

R₁₃, R₁₄, R₁₅ and R₁₆, which may be identical or different, represent aliphatic, alicyclic or arylaliphatic radicals containing from 1 to 20 carbon atoms or lower hydroxyalkylaliphatic radicals, or alternatively R₁₃, R₁₄, R₁₅ and R₁₆, together or separately, constitute, with the nitrogen atoms to which they are attached, heterocycles optionally containing a second hetero atom other than nitrogen, or alternatively R₁₃, R₁₄, R₁₅ and R₁₆ represent a linear or branched C₁-C₆ alkyl radical substituted with a nitrile, ester, acyl or amide group or a group —CO—O—R₁₇-D or —CO—NH—R₁₇-D where R₁₇ is an alkylene and D is a quaternary ammonium group;

A₁ and B₁ represent polymethylene groups containing from 2 to 20 carbon atoms which may be linear or branched, saturated or unsaturated, and which may contain, linked to or intercalated in the main chain, one or more aromatic rings or one or more oxygen or sulfur atoms or sulfoxide, sulfone, disulfide, amino, alkylamino, hydroxyl, quaternary ammonium, ureido, amide or ester groups, and

X⁻ denotes an anion derived from a mineral or organic acid;

A₁, R₁₃ and R₁₅ can form, with the two nitrogen atoms to which they are attached, a piperazine ring; in addition, if A₁ denotes a linear or branched, saturated or unsaturated alkylene or hydroxyalkylene radical, B₁ can also denote a group (CH₂)ₙ—CO-D-OC—(CH₂)ₙ— in which D denotes:

a) a glycol residue of formula: —O-Z-O—, where Z denotes a linear or branched hydrocarbon-based radical or a group corresponding to one of the following formulae:

—(CH₂—CH₂—O)ₓ—CH₂—CH₂—

—[CH₂—CH(CH₃)—O]_{y}—CH₂—CH(CH₃)—

where x and y denote an integer from 1 to 4, representing a defined and unique degree of polymerization or any number from 1 to 4 representing an average degree of polymerization;

b) a bis-secondary diamine residue such as a piperazine derivative;

c) a bis-primary diamine residue of formula: —NH—Y—NH—, where Y denotes a linear or branched hydrocarbon-based radical, or alternatively the divalent radical

—CH₂—CH₂—S—S—CH₂—CH₂—;

d) a ureylene group of formula: —NH—CO—NH—.

Preferably, X⁻ is an anion such as chloride or bromide.

These polymers generally have a number-average molecular mass of between 1000 and 100 000.

Polymers of this type are described in particular in French patents 2 320 330, 2 270 846, 2 316 271, 2 336 434 and 2 413 907 and U.S. Pat. Nos. 2,273,780, 2,375,853, 2,388,614, 2,454,547, 3,206,462, 2,261,002, 2,271,378, 3,874,870, 4,001,432, 3,929,990, 3,966,904, 4,005,193, 4,025,617, 4,025,627, 4,025,653, 4,026,945 and 4,027,020.

It is more particularly possible to use polymers that consist of repeating units corresponding to the formula:

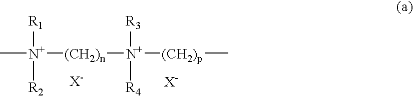

in which R₁, R₂, R₃ and R₄, which may be identical or different, denote an alkyl or hydroxyalkyl radical containing from 1 to 4 carbon atoms approximately, n and p are integers ranging from 2 to 20 approximately, and X⁻ is an anion derived from a mineral or organic acid.

A compound of formula (a) that is particularly preferred is the compound for which R₁, R₂, R₃ and R₄ represent a methyl radical and n=3, p=6 and X=Cl, referred to as hexadimethrine chloride according to the INCI nomenclature (CTFA).

(11) polyquaternary ammonium polymers consisting of units of formula (XIII):

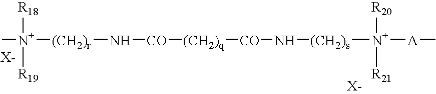

in which formula:

R₁₈, R₁₉, R₂₀ and R₂₁, which may be identical or different, represent a hydrogen atom or a methyl, ethyl, propyl, β-hydroxyethyl, β-hydroxypropyl or —CH₂CH₂(OCH₂CH₂)ₚOH radical,

where p is equal to 0 or to an integer between 1 and 6, with the proviso that R₁₈, R₁₉, R₂₀ and R₂₁ do not simultaneously represent a hydrogen atom,

r and s, which may be identical or different, are integers ranging from 1 to 6,

q is equal to 0 or to an integer ranging from 1 to 34,

X⁻ denotes an anion such as a halide,

A denotes a divalent radical or preferably represents —CH₂—CH₂—O—CH₂—CH₂—.

Such compounds are described in particular in patent application EP-A-122 324.

Among these products, mention may be made, for example, of Mirapol® A 15, Mirapol® AD1, Mirapol® AZ1 and Mirapol® 175 sold by the company Miranol.

(12) Quaternary polymers of vinylpyrrolidone and of vinylimidazole, such as, for example, the products sold under the names Luviquat® FC 905, FC 550 and FC 370 by the company BASF.

(13) Polyamines such as Polyquart® H sold by Henkel, which is given under the reference name “Polyethylene glycol (15) tallow polyamine” in the CTFA dictionary.

(14) Crosslinked methacryloyloxy(C₁-C₄)alkyltri(C₁-C₄)alkylammonium salt polymers such as the polymers obtained by homopolymerization of dimethylaminoethyl methacrylate quaternized with methyl chloride, or by copolymerization of acrylamide with dimethylaminoethyl methacrylate quaternized with methyl chloride, the homo- or copolymerization being followed by crosslinking with a compound containing olefinic unsaturation, in particular methylenebisacrylamide. A crosslinked acrylamide/methacryloyloxyethyltrimethylammonium chloride copolymer (20/80 by weight) in the form of a dispersion containing 50% by weight of said copolymer in mineral oil can be used more particularly. This dispersion is sold under the name Salcare® SC 92 by the company Allied Colloids. A crosslinked methacryloyloxyethyltrimethylammonium chloride homopolymer containing about 50% by weight of the homopolymer in mineral oil or in a liquid ester can also be used. These dispersions are sold under the names Salcare® SC 95 and Salcare® SC 96 by the company Allied Colloids.

Other cationic polymers that can be used in the context of the invention are cationic proteins or cationic protein hydrolyzates, polyalkyleneimines, in particular polyethyleneimines, polymers containing vinylpyridine or vinylpyridinium units, condensates of polyamines and of epichlorohydrin, quaternary polyureylenes and chitin derivatives.

Among all the cationic polymers that may be used in the context of the present invention, it is preferred to use quaternary cellulose ether derivatives such as the products sold under the name JR 400 by the company Union Carbide Corporation, cationic cyclopolymers, in particular the dimethyldiallylammonium chloride homopolymers or copolymers sold under the names Merquat 100, Merquat 550 and Merquat S by the company Calgon, and quaternary polymers of vinylpyrrolidone and of vinylimidazole, and mixtures thereof.

The silicones that may be used in accordance with the invention are in particular polyorganosiloxanes that are insoluble in the composition and that may be in the form of oils, waxes, resins or gums.

The organopolysiloxanes are defined in greater detail in Walter Noll's “Chemistry and Technology of Silicones” (1968) Academic Press. They can be volatile or nonvolatile.

When they are volatile, the silicones are more particularly chosen from those having a boiling point of between 60° C. and 260° C., and even more particularly from:

(i) cyclic silicones containing from 3 to 7 and preferably 4 to 5 silicon atoms. These are, for example, octamethylcyclotetrasiloxane sold in particular under the name Volatile Silicone 7207 by Union Carbide or Silbione 70045 V 2 by Rhône-Poulenc, decamethylcyclopentasiloxane sold under the name Volatile Silicone 7158 by Union Carbide, and Silbione 70045 V 5 by Rhône-Poulenc, and mixtures thereof.

Mention may also be made of cyclocopolymers of the dimethylsiloxanes/methylalkylsiloxane type, such as Volatile Silicone FZ 3109 sold by the company Union Carbide, having the chemical structure:

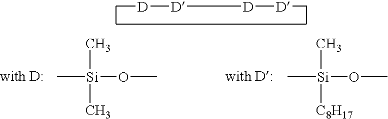

Mention may also be made of mixtures of cyclic silicones with organosilicon compounds, such as the mixture of octamethylcyclotetrasiloxane and tetratrimethylsilylpentaerythritol (50/50) and the mixture of octamethylcyclotetrasiloxane and oxy-1,1′-bis(2,2,2′,2′,3,3′-hexatrimethylsilyloxy)neopentane;

(ii) linear volatile silicones containing 2 to 9 silicon atoms and having a viscosity of less than or equal to 5×10-6 m²/s at 25° C. An example is decamethyltetrasiloxane sold in particular under the name SH 200 by the company Toray Silicone. Silicones belonging to this category are also described in the article published in Cosmetics and Toiletries, Vol. 91, Jan. 76, pp. 27-32, Todd & Byers “Volatile Silicone Fluids for Cosmetics”.

Nonvolatile silicones, and more particularly polyalkylsiloxanes, polyarylsiloxanes, polyalkylarylsiloxanes, silicone gums and resins, polyorganosiloxanes modified with organofunctional groups, and mixtures thereof, are preferably used.

These silicones are more particularly chosen from polyalkylsiloxanes, among which mention may be made mainly of polydimethylsiloxanes containing trimethylsilyl end groups having a viscosity of from 5×10⁻⁶ to 2.5 m²/s at 25° C. and preferably 1×10⁻⁵ to 1 m²/s. The viscosity of the silicones is measured, for example, at 25° C. according to ASTM standard 445 Appendix C.

Among these polyalkylsiloxanes, mention may be made, in a nonlimiting manner, of the following commercial products:

the Silbione oils of the 47 and 70 047 series or the Mirasil oils sold by Rhône-Poulenc, such as, for example, the oil 70 047 V 500 000;

the oils of the Mirasil series sold by the company Rhône-Poulenc;

the oils of the 200 series from the company Dow Corning, such as, more particularly, DC200 with a viscosity of 60 000 cSt;

the Viscasil oils from General Electric and certain oils of the SF series (SF 96, SF 18) from General Electric.

Mention may also be made of polydimethylsiloxanes containing dimethylsilanol end groups (Dimethiconol according to the CTFA name) such as the oils of the 48 series from the company Rhône-Poulenc.

In this category of polyalkylsiloxanes, mention may also be made of the products sold under the names Abil Wax 9800 and 9801 by the company Goldschmidt, which are poly(C₁-C₂₀)alkylsiloxanes.

The polyalkylarylsiloxanes are chosen particularly from linear and/or branched polydimethylmethylphenylsiloxanes or polydimethyldiphenylsiloxanes, with a viscosity of from 1×10⁻⁵ to 5×10⁻² m²/s at 25° C.

Among these polyalkylarylsiloxanes, mention may be made, by way of example, of the products sold under the following names:

the Silbione oils of the 70 641 series from Rhône-Poulenc; the oils of the Rhodorsil 70 633 and 763 series from Rhône-Poulenc;

the oil Dow Corning 556 Cosmetic Grade Fluid from Dow Corning;

the silicones of the PK series from Bayer, such as the product PK20;

the silicones of the PN and PH series from Bayer, such as the products PN1000 and PH1000;

certain oils of the SF series from General Electric, such as SF 1023, SF 1154, SF 1250 and SF 1265.

The silicone gums that can be used in accordance with the invention are, in particular, polydiorganosiloxanes having high number-average molecular masses of between 200 000 and 1 000 000, used alone or as a mixture in a solvent. This solvent can be chosen from volatile silicones, polydimethylsiloxane (PDMS) oils, polyphenylmethylsiloxane (PPMS) oils, isoparaffins, polyisobutylenes, methylene chloride, pentane, dodecane and tridecanes, or mixtures thereof.

Mention may be made more particularly of the following products:

polydimethylsiloxane,

polydimethylsiloxane/methylvinylsiloxane gums,

polydimethylsiloxane/diphenylsiloxane,

polydimethylsiloxane/phenylmethylsiloxane,

polydimethylsiloxane/diphenylsiloxane/methylvinylsiloxane.

Products that can be used more particularly in accordance with the invention are mixtures such as:

mixtures formed from a polydimethylsiloxane hydroxylated at the chain end (referred to as dimethiconol according to the nomenclature in the CTFA dictionary) and from a cyclic polydimethylsiloxane (referred to as cyclomethicone according to the nomenclature in the CTFA dictionary), such as the product Q2 1401 sold by the company Dow Corning;

mixtures formed from a polydimethylsiloxane gum with a cyclic silicone, such as the product SF 1214 Silicone Fluid from the company General Electric; this product is an SF 30 gum corresponding to a dimethicone, having a number-average molecular weight of 500 000, dissolved in the oil SF 1202 Silicone Fluid corresponding to decamethylcyclopentasiloxane;

mixtures of two PDMSS of different viscosities, and more particularly of a PDMS gum and a PDMS oil, such as the product SF 1236 from the company General Electric. The product SF 1236 is a mixture of an SE 30 gum defined above, having a viscosity of 20 m²/s, and an SF 96 oil, with a viscosity of 5×10⁻⁶ m²/s. This product preferably contains 15% SE 30 gum and 85% SF 96 oil.

The organopolysiloxane resins that can be used in accordance with the invention are crosslinked siloxane systems containing the following units: R₂SiO_{2/2}, R₃SiO_{1/2}, RSiO_{3/2} and SiO_{4/2} in which R represents a hydrocarbon-based group containing 1 to 16 carbon atoms or a phenyl group. Among these products, those particularly preferred are the ones in which R denotes a C₁-C₄ lower alkyl radical, more particularly methyl, or a phenyl radical.

Among these resins, mention may be made of the product sold under the name Dow Corning 593 or those sold under the names Silicone Fluid SS 4230 and SS 4267 by the company General Electric, which are silicones of dimethyl/trimethyl siloxane structure.

Mention may also be made of the trimethyl siloxysilicate type resins sold in particular under the names X22-4914, X21-5034 and X21-5037 by the company Shin-Etsu.

The organomodified silicones that can be used in accordance with the invention are silicones as defined above and containing in their structure one or more organofunctional groups attached via a hydrocarbon-based radical.

Among the organomodified silicones, mention may be made of polyorganosiloxanes comprising:

polyethyleneoxy and/or polypropyleneoxy groups optionally comprising C₆-C₂₄ alkyl groups, such as the products known as dimethicone copolyol sold by the company Dow Corning under the name DC 1248 or the oils Silwet L 722, L 7500, L 77 and L 711 by the company Union Carbide, and the (C₁₂)alkylmethicone copolyol sold by the company Dow Corning under the name Q2 5200;

substituted or unsubstituted amine groups, such as the products sold under the name GP 4 Silicone Fluid and GP 7100 by the company Genesee, or the products sold under the names Q2 8220 and Dow Corning 929 or 939 by the company Dow Corning. The substituted amine groups are, in particular, C₁-C₄ aminoalkyl groups;

thiol groups such as the products sold under the names GP 72 A and GP 71 from Genesee;

alkoxylated groups such as the product sold under the name Silicone Copolymer F-755 by SWS Silicones and Abil Wax 2428, 2434 and 2440 by the company Goldschmidt;

hydroxylated groups such as the polyorganosiloxanes containing a hydroxyalkyl function, described in French patent application FR-A-85/16334,

corresponding to formula (XIV):

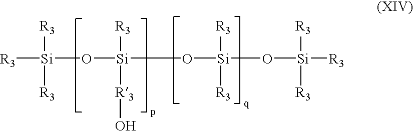

in which the radicals R₃, which may be identical or different, are chosen from methyl and phenyl radicals; at least 60 mol % of the radicals R₃ denoting methyl; the radical R₁₃ is a C₂-C₁₈ divalent hydrocarbon-based alkylene chain unit; p is between 1 and 30 inclusive; q is between 1 and 150 inclusive;

acyloxyalkyl groups such as, for example, the polyorganosiloxanes described in patent U.S. Pat. No. 4,957,732 and corresponding to formula (XV):

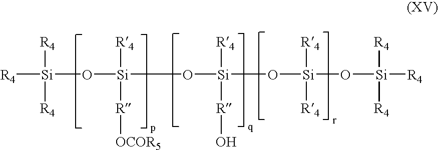

in which:

R₄ denotes a methyl, phenyl, —OCOR₅ or hydroxyl group, one of the radicals R₄ per silicon atom possibly being OH;

R′₄ denotes methyl or phenyl; at least 60 mol % of all the radicals R₄ and R′₄ denoting methyl;

R₅ denotes C₈-C₂₀ alkyl or alkenyl;

R″ denotes a C₂-C₁₈ linear or branched divalent hydrocarbon-based alkylene radical;

r is between 1 and 120 inclusive;

p is between 1 and 30;

q is equal to 0 or is less than 0.5 p, p+q being between 1 and 30; the polyorganosiloxanes of formula (XV) may contain groups:

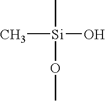

in proportions not exceeding 15% of the sum p+q+r.

anionic groups of carboxylic type, such as, for example, in the products described in patent EP 186 507 from the company Chisso Corporation, or of alkylcarboxylic type, such as those present in the product X-22-3701E from the company Shin-Etsu; 2-hydroxyalkyl sulfonate; 2-hydroxyalkyl thiosulfate such as the products sold by the company Goldschmidt under the names Abil S201 and Abil S255;

hydroxyacylamino groups, such as the polyorganosiloxanes described in patent application EP 342 834. Mention may be made, for example, of the product Q2-8413 from the company Dow Corning.

According to the invention, it is also possible to use silicones comprising a polysiloxane portion and a portion consisting of a nonsilicone organic chain, one of the two portions constituting the main chain of the polymer, the other being grafted onto said main chain. These polymers are described, for example, in patent applications EP-A-412 704, EP-A-412 707, EP-A-640 105, WO 95/00578, EP-A-582 152 and WO 93/23009 and U.S. Pat. Nos. 4,693,935, 4,728,571 and 4,972,037. These polymers are preferably anionic or nonionic.

Such polymers are, for example, copolymers that can be obtained by free-radical polymerization starting with a monomer mixture consisting of:

a) 50 to 90% by weight of tert-butyl acrylate;

b) 0 to 40% by weight of acrylic acid;

c) 5 to 40% by weight of silicone macromer of formula:

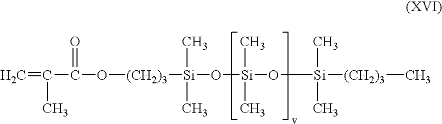

with v being a number ranging from 5 to 700; the weight percentages being calculated relative to the total weight of the monomers.

Other examples of grafted silicone polymers are, in particular, polydimethylsiloxanes (PDMS) onto which are grafted, via a connecting chain unit of thiopropylene type, mixed polymer units of poly(meth)acrylic acid type and of polyalkyl (meth)acrylate type and polydimethylsiloxanes (PDMS) onto which are grafted, via a connecting chain unit of thiopropylene type, polymer units of polyisobutyl (meth)acrylate type.

According to the invention, all of the silicones can also be used in the form of emulsions, nanoemulsions or microemulsions.

The polyorganosiloxanes that are particularly preferred in accordance with the invention are:

nonvolatile silicones chosen from the family of polyalkylsiloxanes containing trimethylsilyl end groups, such as oils having a viscosity of between 0.2 and 2.5 m²/s at 25° C., such as the oils of the DC200 series from Dow Corning, in particular that with a viscosity of 60 000 cSt, of the Silbione 70047 and 47 series and more particularly the oil 70 047 V 500 000, which are sold by the company Rhône-Poulenc, polyalkylsiloxanes containing dimethylsilanol end groups, such as dimethiconols, or polyalkylarylsiloxanes such as the oil Silbione 70641 V 200 sold by the company Rhône-Poulenc;

the organopolysiloxane resin sold under the name Dow Corning 593;

polysiloxanes containing amine groups, such as amodimethicones or trimethylsilylamodimethicones.

The cationic proteins or protein hydrolyzates are, in particular, chemically modified polypeptides bearing quaternary ammonium groups at the end of the chain or grafted thereto. Their molecular mass can range, for example, from 1500 to 10 000 and in particular from 2000 to 5000 approximately. Among these compounds, mention may be made in particular of:

collagen hydrolyzates bearing triethylammonium groups, such as the products sold under the name Quat-Pro E by the company Maybrook and referred to in the CTFA dictionary as “Triethonium Hydrolyzed Collagen Ethosulfate”;

collagen hydrolyzates bearing trimethylammonium and trimethylstearylammonium chloride groups, sold under the name Quat-Pro S by the company Maybrook and referred to in the CTFA dictionary as “Steartrimonium Hydrolyzed Collagen”;

animal protein hydrolyzates bearing trimethylbenzylammonium groups such as the products sold under the name Crotein BTA by the company Croda and referred to in the CTFA dictionary as “Benzyltrimonium hydrolyzed animal protein”;

protein hydrolyzates bearing, on the polypeptide chain, quaternary ammonium groups containing at least one alkyl radical having from 1 to 18 carbon atoms.

Among these protein hydrolyzates, mention may be made, inter alia, of:

Croquat L in which the quaternary ammonium groups contain a C₁₂ alkyl group;

Croquat M in which the quaternary ammonium groups contain C₁₀-C₁₈ alkyl groups;

Croquat S in which the quaternary ammonium groups contain a C₁₈ alkyl group;

Crotein Q in which the quaternary ammonium groups contain at least one alkyl group having from 1 to 18 carbon atoms.

These various products are sold by the company Croda.

Other quaternized proteins or hydrolyzates are, for example, those corresponding to the formula:

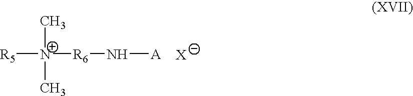

in which X⁻ is an anion of an organic or mineral acid, A denotes a protein residue derived from hydrolyzates of a protein, especially of collagen, R₅ denotes a lipophilic group containing up to 30 carbon atoms and R₆ represents an alkylene group having 1 to 6 carbon atoms. Mention may be made, for example, of the products sold by the company Inolex under the name Lexein QX 3000, referred to in the CTFA dictionary as “Cocotrimonium Collagen Hydrolysate”.

Mention may also be made of quaternized plant proteins such as wheat, corn or soybean proteins: as quaternized wheat proteins, mention may be made of those sold by the company Croda under the names Hydrotriticum WQ or QM, referred to in the CTFA dictionary as “Cocodimonium Hydrolysed Wheat Protein”, Hydrotriticum QL, referred to in the CTFA dictionary as “Lauridimonium Hydrolysed Wheat Protein” or Hydrotriticum QS, referred to in the CTFA dictionary as “Steardimonium Hydrolysed Wheat Protein”.

According to the present invention, the compounds of ceramide type are in particular natural or synthetic ceramides and/or glycoceramides and/or pseudoceramides and/or neoceramides.

Compounds of ceramide type are described, for example, in patent applications DE 4 424 530, DE 4 424 533, DE 4 402 929, DE 4 420 736, WO 95/23807, WO 94/07844, EP-A-0 646 572, WO 95/16665, FR-2 673 179, EP-A-0 227 994, WO 94/07844, WO 94/24097 and WO 94/10131, the teachings of which are included herein by way of reference.

Compounds of ceramide type that are particularly preferred according to the invention are, for example:

2-N-linoleoylaminooctadecane-1,3-diol,

2-N-oleoylaminooctadecane-1,3-diol,

2-N-palmitoylaminooctadecane-1,3-diol,

2-N-stearoylaminooctadecane-1,3-diol,

2-N-behenoylaminooctadecane-1,3-diol,

2-N-[2-hydroxypalmitoyl]aminooctadecane-1,3-diol,

2-N-stearoylaminooctadecane-1,3,4-triol and in particular N-stearoylphytosphingosine,

2-N-palmitoylaminohexadecane-1,3-diol,

bis(N-hydroxyethyl-N-cetyl)malonamide,

N-(2-hydroxyethyl)-N-(3-cetyloxy-2-hydroxypropyl)cetylamide,

N-docosanoyl-N-methyl-D-glucamine,

or mixtures of these compounds.

It is also possible to use cationic surfactants, among which mention may be made in particular of: optionally polyoxyalkylenated primary, secondary or tertiary fatty amine salts; quaternary ammonium salts; imidazoline derivatives; or amine oxides of cationic nature.

Examples of quaternary ammonium salts include:

those of general formula (XVIII) below:

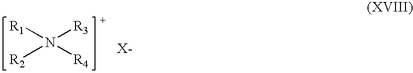

in which the radicals R₁ to R₄, which may be identical or different, represent a linear or branched aliphatic radical containing from 1 to 30 carbon atoms, or an aromatic radical such as aryl or alkylaryl. The aliphatic radicals can comprise hetero atoms such as, in particular, oxygen, nitrogen, sulfur or halogens. The aliphatic radicals are chosen, for example, from alkyl, alkoxy, polyoxy(C₂-C₆) alkylene, alkylamide, (C₁₂-C₂₂)alkylamido(C₂-C₆)alkyl, (C₁₂-C₂₂)alkylacetate and hydroxyalkyl radicals, comprising from about 1 to 30 carbon atoms; X⁻ is an anion chosen from the group of halides, phosphates, acetates, lactates, (C₂-C₆)alkyl sulfates and alkyl or alkylaryl sulfonates;

quaternary ammonium salts of imidazolinium, such as, for example, the salt of formula (XIX) below:

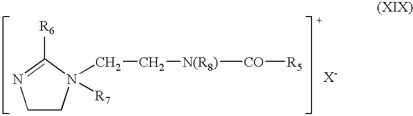

in which R₅ represents an alkenyl or alkyl radical containing from 8 to 30 carbon atoms, for example tallow fatty acid derivatives, R₆ represents a hydrogen atom, a C₁-C₄ alkyl radical or an alkenyl or alkyl radical containing from 8 to 30 carbon atoms, R₇ represents a C₁-C₄ alkyl radical, R₈ represents a hydrogen atom or a C₁-C₄ alkyl radical, X⁻ is an anion chosen from the group of halides, phosphates, acetates, lactates, alkyl sulfates and alkyl or alkylaryl sulfonates. R₅ and R₆ preferably denote a mixture of alkenyl or alkyl radicals containing from 12 to 21 carbon atoms, for example tallow fatty acid derivatives, R₇ denotes a methyl radical and R₈ denotes a hydrogen atom. Such a product is sold, for example, under the name Rewoquat W 75 by the company Rewo;

diquaternary ammonium salts of formula (XX):

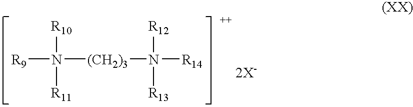

in which R₉ denotes an aliphatic radical containing from about 16 to 30 carbon atoms, R₁₀, R₁₁, R₁₂, R₁₃ and R₁₄, which may be identical or different, are chosen from hydrogen or an alkyl radical containing from 1 to 4 carbon atoms, and X⁻ is an anion chosen from the group of halides, acetates, phosphates, nitrates and methyl sulfates. Such diquaternary ammonium salts in particular comprise propane tallow diammonium dichloride;

quaternary ammonium salts containing at least one ester function.

The quaternary ammonium salts containing at least one ester function that may be used according to the invention are, for example, those of formula (XXI) below:

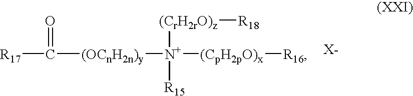

in which:

R₁₅ is chosen from C₁-C₆ alkyl radicals and C₁-C₆ hydroxyalkyl or dihydroxyalkyl radicals;

R₁₆ is chosen from:

a radical

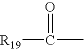

linear or branched, saturated or unsaturated C₁-C₂₂ hydrocarbon-based radicals R₂₀,

a hydrogen atom,

R₁₈ is chosen from:

a radical

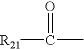

linear or branched, saturated or unsaturated C₁-C₆ hydrocarbon-based radicals R₂₂,

a hydrogen atom,

R₁₇, R₁ ₉ and R₂₁, which may be identical or different, are chosen from linear or branched, saturated or unsaturated C₇-C₂₁ hydrocarbon-based radicals;

n, p and r, which may be identical or different, are integers ranging from 2 to 6;

y is an integer ranging from 1 to 10;

x and z, which may be identical or different, are integers ranging from 0 to 10;

X⁻ is a simple or complex, organic or inorganic anion;

with the proviso that the sum x+y+z is from 1 to 15, that when x is 0, then R₁₆ denotes R₂₀ and that when z is 0, then R₁ denotes R₂₂.

The R₁₅ alkyl radicals may be linear or branched and more particularly linear.

R₁₅ preferably denotes a methyl, ethyl, hydroxyethyl or dihydroxypropyl radical and more particularly a methyl or ethyl radical.

The sum x+y+z is advantageously from 1 to 10.

When R₁₆ is a hydrocarbon-based radical R₂₀, it may be long and contain from 12 to 22 carbon atoms, or short and contain from 1 to 3 carbon atoms.

When R₁₈ is a hydrocarbon-based radical R₂₂, it preferably contains 1 to 3 carbon atoms.

R₁₇, R₁₉ and R₂₁, which may be identical or different, are advantageously chosen from linear or branched, saturated or unsaturated C₁ₗ-C₂, hydrocarbon-based radicals, and more particularly from linear or branched, saturated or unsaturated, C₁₁-C₂₁ alkyl and alkenyl radicals.

x and z, which may be identical or different, are preferably 0 or 1.

y is advantageously equal to 1.

n, p and r, which may be identical or different, are preferably 2 or 3 and even more particularly are equal to 2.

The anion is preferably a halide (chloride, bromide or iodide) or an alkyl sulfate, more particularly methyl sulfate. However, methanesulfonate, phosphate, nitrate, tosylate, an anion derived from an organic acid, such as acetate or lactate, or any other anion that is compatible with the ammonium containing an ester function, may be used.

The anion X⁻ is even more particularly chloride or methyl sulfate.

The ammonium salts more particularly used in the composition according to the invention are those of formula (XXI) in which:

R₁₅ denotes a methyl or ethyl radical,

x and y are equal to 1;

z is equal to 0 or 1;

n, p and r are equal to 2;

R₁₆ is chosen from:

a radical

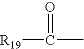

methyl, ethyl or C₁₄-C₂₂ hydrocarbon-based radicals;

a hydrogen atom;

R₁₈ is chosen from:

a radical

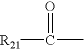

a hydrogen atom;

R₁₇, R₁₉ and R₂₁, which may be identical or different, are chosen from linear or branched, saturated or unsaturated C₁₃-C₁₇ hydrocarbon-based radicals and preferably from linear or branched, saturated or unsaturated C₁₃-C₁₇ alkyl and alkenyl radicals.

The hydrocarbon-based radicals are advantageously linear.

Examples that may be mentioned include the compounds of formula (XXI) such as the diacyloxyethyl-dimethylammonium, diacyloxyethylhydroxyethylmethylammonium, monoacyloxyethyldihydroxyethylmethylammonium, triacyloxyethylmethylammonium and monoacyloxyethyl-hydroxyethyldimethylammonium salts (chloride or methyl sulfate in particular), and mixtures thereof. The acyl radicals preferably contain 14 to 18 carbon atoms and are obtained more particularly from a plant oil such as palm oil or sunflower oil. When the compound contains several acyl radicals, these radicals may be identical or different.

These products are obtained, for example, by direct esterification of triethanolamine, triisopropanolamine, an alkyldiethanolamine or an alkyldiisopropanolamine, which are optionally oxyalkylenated, with fatty acids or with fatty acid mixtures of plant or animal origin, or by transesterification of the methyl esters thereof. This esterification is followed by a quaternization using an alkylating agent such as an alkyl halide (preferably a methyl or ethyl halide), a dialkyl sulfate (preferably dimethyl or diethyl sulfate), methyl methanesulfonate, methyl para-toluenesulfonate, glycol chlorohydrin or glycerol chlorohydrin.

Such compounds are sold, for example, under the names Dehyquart by the company Henkel, Stepanquat by the company Stepan, Noxamium by the company CECA or Rewoquat WE 18 by the company Rewo-Witco.

It is also possible to use the ammonium salts containing at least one ester function that are described in patents U.S. Pat. No. 4,874,554 and U.S. Pat. No. 4,137,180.

Among the quaternary ammonium salts of formula (XVIII), the ones that are preferred are, on the one hand, tetraalkylammonium chlorides such as, for example, dialkyldimethylammonium chlorides or alkyltrimethylammonium chlorides, in which the alkyl radical contains from about 12 to 22 carbon atoms, in particular behenyltrimethylammonium chloride, distearyldimethyl-ammonium chloride, cetyltrimethylammonium chloride, or benzyldimethylstearylammonium chloride, or, on the other hand, palmitylamidopropyltrimethylammonium chloride or stearamidopropyldimethyl(myristyl acetate)ammonium chloride sold under the name Ceraphyl 70 by the company Van Dyk.

The fatty acids are chosen more particularly from myristic acid, palmitic acid, stearic acid, behenic acid, oleic acid, linoleic acid, linolenic acid and isostearic acid.

The fatty alcohol or fatty acid derivatives are especially carboxylic acid esters, in particular mono-, di- tri- or tetracarboxylic esters.

The monocarboxylic acid esters are, in particular, linear or branched, saturated or unsaturated C₁-C₂₆ aliphatic acid monoesters of linear or branched, saturated or unsaturated, C₁-C₂₆ aliphatic alcohols, the total carbon number of these esters being greater than or equal to 10.

Among the monoesters, mention may be made of dihydroabietyl behenate; octyldodecyl behenate; isocetyl behenate; cetyl lactate; C₁₂-C₁₅ alkyl lactate; isostearyl lactate; lauryl lactate; linoleyl lactate; oleyl lactate; (iso)stearyl octanoate; isocetyl octanoate; octyl octanoate; cetyl octanoate; decyl oleate; isocetyl isostearate; isocetyl laurate; isocetyl stearate; isodecyl octanoate; isodecyl oleate; isononyl isononanoate; isostearyl palmitate; methylacetyl ricinoleate; myristyl stearate; octyl isononanoate; 2-ethylhexyl isononate; octyl palmitate; octyl pelargonate; octyl stearate; octyldodecyl erucate; oleyl erucate; ethyl and isopropyl palmitates, 2-ethylhexyl palmitate, 2-octyldecyl palmitate, alkyl myristates such as isopropyl, butyl, cetyl or 2-octyldodecyl myristate, hexyl stearate, butyl stearate, isobutyl stearate; dioctyl malate, hexyl laurate, 2-hexyldecyl laurate.

C₄-C₂₂ di- or tricarboxylic acid esters of C₁-C₂₂ alcohols and mono-, di- or tricarboxylic acid esters of C₂-C₂₆ di-, tri-, tetra- or pentahydroxy alcohols can also be used.

Mention may be made in particular of: diethyl sebacate; diisopropyl sebacate; diisopropyl adipate; di-n-propyl adipate; dioctyl adipate; diisostearyl adipate; dioctyl maleate; glyceryl undecylenate; octyldodecylstearoyl stearate; pentaerythrityl monoricinoleate; pentaerythrityl tetraisononanoate; pentaerythrityl tetrapelargonate; pentaerythrityl tetraisostearate; pentaerythrityl tetraoctanoate; propylene glycol dicaprylate dicaprate; tridecyl erucate; triisopropyl citrate; triisostearyl citrate; glyceryl trilactate; glyceryl trioctanoate; trioctyldodecyl citrate; trioleyl citrate.

Among the esters mentioned above, it is preferred to use ethyl and isopropyl palmitates, 2-ethylhexyl palmitate, 2-octyldecyl palmitate, alkyl myristates such as isopropyl, butyl, cetyl or 2-octyldodecyl myristate, hexyl stearate, butyl stearate, isobutyl stearate; dioctyl malate, hexyl laurate, 2-hexyldecyl laurate, isononyl isononanoate and cetyl octanoate.

The fluoro oils are, for example, the perfluoropolyethers described in particular in patent application EP-A-486 135 and the fluorohydrocarbon compounds described in particular in patent application WO 93/11103. The teaching of these two patent applications is included in its entirety in the present application by way of reference.

The term “fluorohydrocarbon compounds” denotes compounds whose chemical structure contains a carbon skeleton in which certain hydrogen atoms have been replaced with fluorine atoms.

The fluoro oils can also be fluorocarbons such as fluoroamines, for example perfluorotributylamine, fluorohydrocarbons, for example perfluorodecahydronaphthalene, fluoro esters and fluoro ethers.

The perfluoropolyethers are sold, for example, under the trade names Fomblin by the company Montefluos and Krytox by the company Du Pont.

Among the fluorohydrocarbon compounds, mention may also be made of fluorine-containing fatty acid esters such as the product sold under the name Nofable FO by the company Nippon Oil.

Needless to say, it is possible to use mixtures of conditioners.

According to the invention, the conditioner(s) may represent from 0.001% to 20% by weight, preferably from 0.01% to 10% by weight and more particularly from 0.1% to 3% by weight, relative to the total weight of the final composition.

The compositions of the invention also advantageously contain at least one surfactant, which is generally present in an amount of between 0.1% and 60% by weight approximately, preferably between 1% and 40% and even more preferably between 5% and 30%, relative to the total weight of the composition.

This surfactant may be chosen from anionic, amphoteric and nonionic surfactants, or mixtures thereof.

The surfactants that are suitable for carrying out the present invention are especially the following:

(i) Anionic Surfactant(s):

In the context of the present invention, their nature does not represent a truly critical factor.

Thus, as examples of anionic surfactants which can be used, alone or as mixtures, in the context of the present invention, mention may be made in particular (nonlimiting list) of salts (in particular alkaline salts, especially sodium salts, ammonium salts, amine salts, amino alcohol salts or magnesium salts) of the following compounds: alkyl sulfates, alkyl ether sulfates, alkylamido ether sulfates, alkylarylpolyether sulfates, monoglyceride sulfates; alkyl sulfonates, alkyl phosphates, alkylamide sulfonates, alkylaryl sulfonates, α-olefin sulfonates, paraffin sulfonates; alkyl sulfosuccinates, alkyl ether sulfosuccinates, alkylamide sulfosuccinates; alkyl sulfosuccinamates; alkyl sulfoacetates; alkyl ether phosphates; acyl sarcosinates; acyl isethionates and N-acyltaurates, the alkyl or acyl radical of all of these various compounds preferably containing from 8 to 24 carbon atoms, and the aryl radical preferably denoting a phenyl or benzyl group. Among the anionic surfactants which can also be used, mention may also be made of fatty acid salts such as the salts of oleic, ricinoleic, palmitic and stearic acids, coconut oil acid or hydrogenated coconut oil acid; acyl lactylates in which the acyl radical contains 8 to 20 carbon atoms. Weakly anionic surfactants can also be used, such as alkyl-D-galactosiduronic acids and their salts, as well as polyoxyalkylenated (C₆-C₂₄) alkyl ether carboxylic acids, polyoxyalkylenated (C₆-C₂₄) alkylaryl ether carboxylic acids, polyoxyalkylenated (C₆-C₂₄) alkylamido ether carboxylic acids and their salts, in particular those containing from 2 to 50 ethylene oxide groups, and mixtures thereof.

Among the anionic surfactants, it is preferred according to the invention to use alkyl sulfate salts and alkyl ether sulfate salts and mixtures thereof.

(ii) Nonionic Surfactant(s):

The nonionic surfactants are, themselves also, compounds that are well known per se (see in particular in this respect “Handbook of Surfactants” by M. R. Porter, published by Blackie & Son (Glasgow and London), 1991, pp. 116-178) and, in the context of the present invention, their nature is not a critical feature. Thus, they can be chosen in particular from (nonlimiting list) polyethoxylated, polypropoxylated or polyglycerolated fatty acids, alkylphenols, α-diols or alcohols having a fatty chain containing, for example, 8 to 18 carbon atoms, it being possible for the number of ethylene oxide or propylene oxide groups to range in particular from 2 to 50 and for the number of glycerol groups to range in particular from 2 to 30. Mention may also be made of copolymers of ethylene oxide and of propylene oxide, condensates of ethylene oxide and of propylene oxide with fatty alcohols; polyethoxylated fatty amides preferably having from 2 to 30 mol of ethylene oxide, polyglycerolated fatty amides containing on average 1 to 5, and in particular 1.5 to 4, glycerol groups; polyethoxylated fatty amines preferably having 2 to 30 mol of ethylene oxide; oxyethylenated fatty acid esters of sorbitan having from 2 to 30 mol of ethylene oxide; fatty acid esters of sucrose, fatty acid esters of polyethylene glycol, alkylpolyglycosides, N-alkylglucamine derivatives, amine oxides such as (C₁₀-C₁₄)alkylamine oxides or N-acylaminopropylmorpholine oxides. It will be noted that the alkylpolyglycosides constitute nonionic surfactants that are particularly suitable in the context of the present invention.

(iii) Amphoteric Surfactant(s):

The amphoteric surfactants, whose nature is not a critical feature in the context of the present invention, can be, in particular (nonlimiting list), aliphatic secondary or tertiary amine derivatives in which the aliphatic radical is a linear or branched chain containing 8 to 22 carbon atoms and containing at least one water-soluble anionic group (for example carboxylate, sulfonate, sulfate, phosphate or phosphonate); mention may also be made of (C₈-C₂₀)alkylbetaines, sulfobetaines, (C₈-C₂₀)alkylamido(C₁-C₆)alkylbetaines or (C₈-C₂₀)alkylamido(C₁-C₆)alkylsulfobetaines.

Among the amine derivatives, mention may be made of the products sold under the name Miranol, as described in U.S. Pat. Nos. 2,528,378 and 2,781,354 and having the structures:

R₂—CONHCH₂CH₂—N(R₃) (R₄) (CH₂COO—) (2)

in which: R₂ denotes an alkyl radical derived from an acid R₂—COOH present in hydrolyzed coconut oil, a heptyl, nonyl or undecyl radical, R₃ denotes a β-hydroxyethyl group and R₄ denotes a carboxymethyl group; and

R₅—CONHCH₂CH₂—N(B)(C) (3)

in which:

B represents —CH₂CH₂OX′, C represents —(CH₂)ₙ—Y′, with z=1 or 2,

X′ denotes the —CH₂CH₂—COOH group or a hydrogen atom,

Y′ denotes —COOH or the —CH₂—CHOH—SO₃H radical,

R₅ denotes an alkyl radical of an acid R₉-COOH present in coconut oil or in hydrolyzed linseed oil, an alkyl radical, in particular a C₇, C₉, C₁₁ or C₁₃ alkyl radical, a C₁₇ alkyl radical and its iso form, an unsaturated C₁₇ radical.

These compounds are classified in the CTFA dictionary, 5th edition, 1993, under the names disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, disodium capryloamphodipropionate, lauroamphodipropionic acid, cocoamphodipropionic acid.

By way of example, mention may be made of the cocoamphodiacetate sold under the trade name Miranol C2M concentrate by the company Rhône-Poulenc.

In the compositions in accordance with the invention, mixtures of surfactants are preferably used, and in particular mixtures of anionic surfactants and mixtures of anionic surfactants and of amphoteric or nonionic surfactants. One mixture that is particularly preferred is a mixture consisting of at least one anionic surfactant and of at least one amphoteric surfactant.

The anionic surfactants preferably used are chosen from sodium, triethanolamine or ammonium (C₁₂-C₁₄)alkyl sulfates, sodium, triethanolamine or ammonium (C₁₂-C₁₄)alkyl ether sulfates oxyethylenated with 2.2 mol of ethylene oxide, sodium cocoyl isethionate and sodium α-(C₁₄-C₁₆)olefin sulfonate, and mixtures thereof, with:

either an amphoteric surfactant such as the amine derivatives known as disodium cocoamphodipropionate or sodium cocoamphopropionate sold especially by the company Rhône-Poulenc under the trade name Miranol C2M CONC as an aqueous solution containing 38% active material, or under the name Miranol C32;

or an amphoteric surfactant of zwitterionic type such as alkylbetaines, in particular the cocobetaine sold under the name Dehyton AB 30 as an aqueous solution containing 32% AM by the company Henkel.

The composition of the invention may also contain at least one additive chosen from thickeners, fragrances, nacreous agents, preserving agents, anionic or nonionic polymers, noncationic proteins, noncationic protein hydrolyzates, 18-methyleicosanoic acid, hydroxy acids, polymers other than those of the invention and in particular polyether cationic or nonionic associative polyurethanes, and any other additive conventionally used in cosmetics that does not affect the properties of the compositions according to the invention.

These additives are present in the composition according to the invention in proportions that may range from 0 to 20% by weight relative to the total weight of the composition. The precise amount of each additive is readily determined by a person skilled in the art, depending on its nature and its function.

The compositions in accordance with the invention may be used more particularly for washing or treating keratin materials such as the hair, the skin, the eyelashes, the eyebrows, the nails, the lips or the scalp, and more particularly the hair.

In particular, the compositions according to the invention are detergent compositions such as shampoos, shower gels and bubble baths. In this embodiment of the invention, the compositions comprise at least one washing base, which is generally aqueous.

The surfactant(s) forming the washing base may be chosen, without discrimination, alone or as mixtures, from the anionic, amphoteric and nonionic surfactants as defined above.

The quantity and quality of the washing base are those that are sufficient to give the final composition satisfactory foaming power and/or detergent power.

Thus, according to the invention, the washing base can represent from 4% to 50% by weight, preferably from 6% to 35% by weight and even more preferably from 8% to 25% by weight, relative to the total weight of the final composition.

The pH of the composition applied to the keratin materials is generally between 2 and 11. It is preferably between 3 and 8, and may be adjusted to the desired value by means of acidifying or basifying agents that are well known in the prior art for compositions applied to keratin fibers.

Among the basifying agents that may be mentioned, for example, are aqueous ammonia, alkali metal carbonates, alkanolamines such as monoethanolamine, diethanolamine and triethanolamine and also derivatives thereof, oxyethylenated and/or oxypropylenated hydroxyalkylamines and ethylenediamines, sodium hydroxide, potassium hydroxide and the compounds of formula (XXII) below:

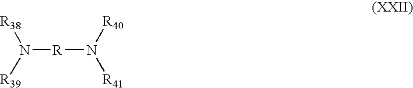

in which R is a propylene residue optionally substituted with a hydroxyl group or a C₁-C₄ alkyl radical; R₃₈, R₃₉, R₄₀ and R₄₁, which may be identical or different, represent a hydrogen atom, a C₁-C₄ alkyl radical or a C₁-C₄ hydroxyalkyl radical.

The acidifying agents are conventionally, for example, mineral or organic acids, for instance hydrochloric acid, orthophosphoric acid, carboxylic acids, for instance tartaric acid, citric acid or lactic acid, or sulfonic acids.

The physiologically and in particular cosmetically acceptable medium may consist solely of water, of a cosmetically acceptable solvent or of a mixture of water and a cosmetically acceptable solvent such as, especially, a C₁-C₄ lower alcohol, for instance ethanol, isopropanol, tert-butanol or n-butanol; alkylene glycols, for instance propylene glycol, and glycol ethers.

A subject of the invention is also a process for treating keratin materials such as the skin or the hair, characterized in that it consists in applying to the keratin materials a cosmetic composition as defined above, optionally followed by rinsing with water.

Thus, this process according to the invention allows holding of the hairstyle and the treatment, care and washing of or removal of makeup from the skin, the hair or any other keratin material.

The compositions of the invention may also be in the form of a rinse-out or leave-in conditioner, permanent-waving, hair-straightening, dyeing or bleaching compositions, or alternatively in the form of rinse-out compositions to be applied before or after dyeing, bleaching, permanent-waving or straightening the hair, or alternatively between the two steps of a permanent-waving or hair-straightening operation.

The compositions of the invention may also be in the form of washing compositions for the skin, in particular in the form of bath or shower solutions or gels or makeup-removing products.

The compositions according to the invention may also be in the form of aqueous or aqueous-alcoholic lotions for skincare and/or haircare.

The cosmetic compositions according to the invention may be in the form of a gel, a milk, a cream, an emulsion, a thickened lotion or a mousse and may be used for the skin, the nails, the eyelashes, the lips and, more particularly, the hair.

The compositions may be packaged in various forms, especially in vaporizers, pump-dispenser bottles or in aerosol containers to allow the composition to be applied in vaporized form or in the form of a mousse. Such packaging forms are indicated, for example, when it is desired to obtain a spray, a lacquer or a mousse for treating the hair.

Throughout the text hereinabove and hereinbelow, the percentages expressed are on a weight basis.

The invention will now be illustrated more fully with the aid of the examples that follow, which cannot be considered as limiting it to the embodiments described. In the examples, AM means active material.

Polymer 1 is a vinylpyrrolidone/dimethylaminopropylmethacrylamide/lauryldimethylmethacrylamido-ammonium chloride terpolymer sold by the company ISP under the reference Polymer ACP-1234.

### EXAMPLE 1

A shampoo composition was prepared:

| Sodium lauryl ether sulfate | 17 | g AM |
|---|---|---|
| containing 2.2 m [symbol] | | |
| of ethylene oxide, at 28% AM | | |
| Cocoylbetaine at 30% AM | 2.5 | g AM |
| Polymer 1 | 1 | g AM |
| Coconut acid monoisopropanolamide | 0.6 | g |
| 2-hydroxy-4-methoxybenzophenone- | 0.1 | g |
| 5-sulfonic a [symbol] (Uvinul | | |
| MS 40 from BASF) | | |
| Fragrance, preserving agents | qs | |
| Demineralized water qs | 100 | g |

Hair treated with this shampoo is smooth, soft and protected against the harmful action of light.

### EXAMPLE 2

The following shampoo composition was prepared:

| | | |
|---|---|---|
| Sodium lauryl ether sulfate | 10 | g AM |
| containing 2.2 m [symbol] of ethylene | | |
| oxide, at 30% AM | | |
| Cocoylbetaine at 30% AM | 4 | g AM |
| Polymer 1 | 0.5 | g AM |
| Polydimethylsiloxane of | 0.5 | g |
| viscosity 300 000 cS | | |
| (Silicone AK300 000 from Wacker) | | |
| Xanthan gum | 1 | g |
| Citric acid qs pH | 7 | |
| Demineralized water qs | 100 | g |

Hair treated with this shampoo is smooth and soft.

### EXAMPLE 3

A conditioner in accordance with the invention, having the composition below, was prepared:

| | | |
|---|---|---|
| Polymer 1 | 0.5 | g AM |
| Behenyltrimethylammonium chloride | 1.5 | g AM |
| Mixture of cetylstearyl alcohol | | |
| and of cetylstearyl alcohol | | |
| oxyethylenated with 33 E [symbol] | | |
| (80/20) | 4 | g |
| Demineralized water qs | 100 | g |

Hair treated with this conditioner is smooth and soft.

### EXAMPLE 4

A conditioner in accordance with the invention, having the composition below, was prepared:

| | | |
|---|---|---|
| Polymer 1 | 0.5 | g AM |
| Behenyltrimethylammonium chloride | 1.5 | g AM |
| N-Oleoyldihydrosphingosine | 0.5 | g |
| Water qs | 100 | g |

Hair treated with this conditioner is smooth and soft.

1. A composition for treating keratin materials, in particular human keratin fibers such as the hair, comprising, in a physiologically and in particular a cosmetically acceptable medium, at least one protecting and/or conditioning agent and at least one cationic poly(alkyl)vinyllactam polymer comprising:
a) at least one monomer of vinyllactam or (C₁-C₅)alkylvinyllactam type;
b) at least one monomer chosen from those of structures (I) and (II): 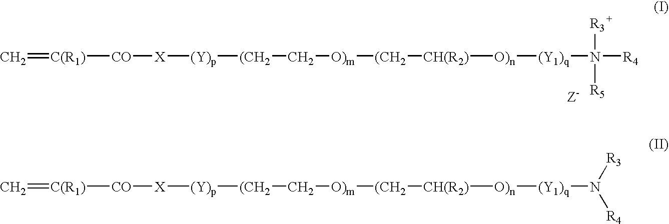
in which:
X denotes an oxygen atom or a radical NR₆,
R₁ and R₆ denote, independently of each other, a hydrogen atom or a linear or branched C₁-C₅ alkyl radical,
R₂ denotes a linear or branched C₁-C₄ alkyl radical,
R₃, R₄ and R₅ denote, independently of each other, a hydrogen atom, a linear or branched C₁-C₃₀ alkyl radical or a radical of formula (III):
—(Y₂)ᵣ—(CH₂—CH(R₇)—O)ₓ—R₈ (III)
Y, Y₁ and Y₂ denote, independently of each other, a linear or branched C₂-C₁₆ alkylene radical,
R₇ denotes hydrogen, a linear or branched C₁-C₄ alkyl radical or a linear or branched C₁-C₄ hydroxyalkyl radical,
R₈ denotes a hydrogen atom or a linear or branched C₁-C₃₀ alkyl radical,
p, q and r denote, independently of each other, either the value 0 or the value 1,
m and n denote, independently of each other, an integer ranging from 0 to 100,
x denotes an integer ranging from 1 to 100,
Z denotes an organic or mineral acid anion,
with the proviso that:
at least one of the substituents R₃, R₄, R₅ or R₈ denotes a linear or branched C₉-C₃₀ alkyl radical,
if m or n is other than 0, then q is equal to 1,
if m or n are equal to 0, then either p or q is equal to 0.

2. The composition as claimed in claim 1, characterized in that the vinyllactam or alkylvinyllactam monomer is a compound of structure (IV): 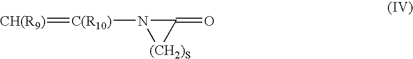
in which:
s denotes an integer ranging from 3 to 6,
R₉ denotes a hydrogen atom or a C₁-C₅ alkyl radical,
R₁₀ denotes a hydrogen atom or a C₁-C₅ alkyl radical,
with the proviso that at least one of the radicals R₉ and R₁₀ denotes a hydrogen atom.

3. The composition as claimed in claim 2, characterized in that the monomer of formula (IV) is vinylpyrrolidone.

4. The composition as claimed in any one of claims 1 to 3, characterized in that R₃, R₄ and R₅ in formulae (I) and/or (II) denote, independently of each other, a hydrogen atom or a linear or branched C₁-C₃₀ alkyl radical.

5. The composition as claimed in any one of claims 1 to 4, characterized in that the monomer b) is a monomer of formula (I).

6. The composition as claimed in any one of claims 1 to 5, characterized in that the numbers m and n are equal to 0.

7. The composition as claimed in any one of claims 1 to 6, characterized in that the counterion Z⁻ of the monomers of formula (I) is chosen from halide ions, phosphate ions, methosulfate ions and tosylate ions.

8. The composition as claimed in any one of claims 1 to 7, characterized in that the cationic polymer(s) according to the invention also contain(s) one or more additional monomer(s), which are preferably cationic or nonionic.

9. The composition as claimed in any one of claims 1 to 8, characterized in that the polymer is a terpolymer comprising:
a) one monomer of formula (IV),
b) one monomer of formula (I) in which p=1, q=O, R₃ and R₄ denote a hydrogen atom or a C₁-C₅ alkyl radical and R₅ denotes a C₉-C₂₄ alkyl radical, and
c) one monomer of formula (II) in which R₃ and R₄ denote a hydrogen atom or a C₁-C₅ alkyl radical.

10. The composition as claimed in claim 9, characterized in that the terpolymer comprises, by weight, 40% to 95% of monomer (a), 0.25% to 50% of monomer (b) and 0.1% to 55% of monomer (c).

11. The composition as claimed in any one of claims 1 to 10, characterized in that the cationic poly(alkyl)vinyllactam polymers are vinylpyrrolidone/dimethylaminopropylmethacrylamide/dodecyldimethylmethacrylamidopropylammonium tosylate terpolymers, vinylpyrrolidone/dimethylaminopropylmethacrylamide/cocoyldimethylmethacrylamidopropylammonium tosylate terpolymers, or vinylpyrrolidone/dimethylaminopropylmethacrylamide/lauryldimethylmethacrylamidopropylammonium tosylate or chloride terpolymers.

12. The composition as claimed in any one of claims 1 to 11, characterized in that the weight-average molecular mass of the cationic polymers is between 500 and 20 000 000, preferably between 200 000 and 2 000 000 and even more preferably between 400 000 and 800 000.

13. The composition as claimed in any one of the preceding claims, characterized in that the cationic poly(alkyl)vinyllactam polymers are used in an amount ranging from 0.01% to 10% by weight relative to the total weight of the composition.

14. The composition as claimed in claim 13, characterized in that the cationic poly(alkyl)vinyllactam polymers are used in an amount ranging from 0.1% to 5% by weight relative to the total weight of the composition.

15. The composition as claimed in any one of the preceding claims, characterized in that said agent for protecting keratin materials is chosen from UV-screening agents, free-radical scavengers, antioxidants, vitamins and provitamins.

16. The composition as claimed in the preceding claim, characterized in that said UV-screening agents are chosen from water-soluble, liposoluble or water-insoluble, silicone or nonsilicone organic screening agents and mineral oxide nanoparticles which have optionally been surface-treated.

17. The composition as claimed in the preceding claim, characterized in that said water-soluble organic UV-screening agent is chosen from the group consisting of para-aminobenzoic acid and its salts, anthranilic acid and its salts, salicylic acid and its salts, p-hydroxycinnamic acid and its salts, sulfonic derivatives of benz-x-azoles and salts thereof, sulfonic derivatives of benzophenone and salts thereof, sulfonic derivatives of benzylidenecamphor and salts thereof, benzylidenecamphor derivatives substituted with a quaternary amine and salts thereof, phthalylidene-camphorsulfonic acid derivatives and salts thereof, sulfonic derivatives of benzotriazole, and hydrophilic polymers having, in addition and on account of their chemical nature, UV-photoprotective properties, and mixtures thereof.

18. The composition as claimed in either of claims 16 and 17, characterized in that said liposoluble organic UV-screening agent is chosen from: p-aminobenzoic acid derivatives, such as p-aminobenzoic acid esters or amides; salicylic acid derivatives such as salicylic acid esters; benzophenone derivatives; dibenzoylmethane derivatives; diphenylacrylate derivatives; benzofuran derivatives; polymeric UV-screening agents containing one or more organosilicon residues; cinnamic acid esters; camphor derivatives; trianilino-s-triazine derivatives; urocanic acid ethyl ester; benzotriazoles; hydroxyphenyltriazine derivatives; bis-resorcinoldialkylaminotriazines; and mixtures thereof.

19. The composition as claimed in claim 18, characterized in that said liposoluble UV-screening agent is chosen from: octyl salicylate; 2-hydroxy-4-methoxybenzophenone; 4-tert-butyl-4′-methoxydibenzoylmethane; octocrylene; 2-ethylhexyl 4-methoxycinnamate; and the compound of formula (V):

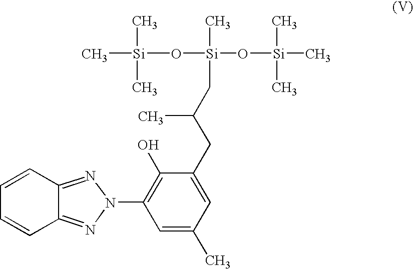

20. The composition as claimed in any one of the preceding claims, characterized in that the conditioners are chosen from synthetic oils, mineral oils, plant oils, fluoro oils or perfluoro oils, natural or synthetic waxes, silicones, cationic polymers other than the poly(alkyl)vinyllactams defined in claims 1 to 12, compounds of ceramide type, cationic surfactants, fatty amines, fatty acids and derivatives thereof, and also mixtures of these various compounds.

21. The composition as claimed in claim 20, characterized in that the synthetic oils are polyolefins of hydrogenated or nonhydrogenated polybutene type, or of hydrogenated or nonhydrogenated polydecene type.

22. The composition as claimed in claim 20, characterized in that the cationic polymers are chosen from those containing units comprising primary, secondary, tertiary and/or quaternary amine groups that may either form part of the main polymer chain or be borne by a side substituent directly attached thereto.

23. The composition as claimed in either of claims 20 and 22, characterized in that said cationic polymer is chosen from quaternary cellulose ether derivatives, cationic cyclopolymers, cationic polysaccharides, and quaternary polymers of vinylpyrrolidone and of vinylimidazole, and mixtures thereof.

24. The composition as claimed in claim 23, characterized in that said cyclopolymer is chosen from diallyldimethylammonium chloride homopolymers and copolymers of diallyldimethylammonium chloride and of acrylamide.

25. The composition as claimed in claim 23, characterized in that said quaternary cellulose ether derivatives are chosen from hydroxyethylcelluloses that have reacted with an epoxide substituted with a trimethylammonium group.

26. The composition as claimed in claim 23, characterized in that said cationic polysaccharides are chosen from guar gums modified with a 2,3-epoxypropyltrimethylammonium salt.

27. The composition as claimed in claim 20, characterized in that the silicones are chosen from polyorganosiloxanes that are insoluble in the composition.

28. The composition as claimed in either of claims 20 and 27, characterized in that the polyorganosiloxanes are nonvolatile polyorganosiloxanes chosen from polyalkylsiloxanes, polyarylsiloxanes, polyalkylarylsiloxanes, silicone gums and resins, and polyorganosiloxanes modified with organofunctional groups, and mixtures thereof.

29. The composition as claimed in claim 28, characterized in that:
(a) the polyalkylsiloxanes are chosen from:
polydimethylsiloxanes containing trimethylsilyl end groups;
polydimethylsiloxanes containing dimethylsilanol end groups;
poly(C₁-C₂₀)alkylsiloxanes;
(b) the polyalkylarylsiloxanes are chosen from:
linear and/or branched polydimethylmethylphenylsiloxanes or polydimethyldiphenylsiloxanes, with a viscosity of between 1×10⁻⁵ and 5×10⁻² m²/s at 25° C.;
(c) the silicone gums are chosen from polydiorganosiloxanes with number-average molecular masses of between 200 000 and 1 000 000, used alone or in the form of a mixture in a solvent;
(d) the resins are chosen from resins consisting of units:
R₃SiO_{1/2}, R₂SiO_{2/2}, RSiO_{3/2} and SiO_{4/2}
in which R represents a hydrocarbon-based group containing from 1 to 16 carbon atoms or a phenyl group;
(e) the organomodified silicones are chosen from silicones comprising in their structure one or more organofunctional groups attached via a hydrocarbon-based radical.

30. The composition as claimed in claim 29, characterized in that the silicone gums used, alone or in the form of a mixture, are chosen from the following structures:
polydimethylsiloxane,
polydimethylsiloxane/methylvinylsiloxanes,
polydimethylsiloxane/diphenylsiloxane,
polydimethylsiloxane/phenylmethylsiloxane,
polydimethylsiloxane/diphenylsiloxane/methylvinylsiloxanes, and the following mixtures:
mixtures formed from a polydimethylsiloxane hydroxylated at the chain end and from a cyclic polydimethylsiloxane;
mixtures formed from a polydimethylsiloxane gum and from a cyclic silicone; and
mixtures of polydimethylsiloxanes of different viscosities.

31. The composition as claimed in claim 29, characterized in that the organomodified silicones are chosen from polyorganosiloxanes comprising:
a) polyethyleneoxy and/or polypropyleneoxy groups;
b) substituted or unsubstituted amine groups;
c) thiol groups;
d) alkoxylated groups;
e) hydroxyalkyl groups;
f) acyloxyalkyl groups;
g) alkylcarboxylic groups;
h) 2-hydroxyalkyl sulfonate groups;
i) 2-hydroxyalkyl thiosulfonate groups;
j) hydroxyacylamino groups.

32. The composition as claimed in any one of claims 27 to 31, characterized in that the polyorganosiloxanes are chosen from polyalkylsiloxanes containing trimethylsilyl end groups, polyalkylsiloxanes containing dimethylsilanol end groups, polyalkylarylsiloxanes, mixtures of two PDMSs consisting of a gum and an oil of different viscosities, mixtures of organosiloxanes and of cyclic silicones, and organopolysiloxane resins.

33. The composition as claimed in claim 20, characterized in that the compounds of ceramide type are chosen from:
2-N-linoleoylaminooctadecane-1,3-diol,
2-N-oleoylaminooctadecane-1,3-diol,
2-N-palmitoylaminooctadecane-1,3-diol,
2-N-stearoylaminooctadecane-1,3-diol,
2-N-behenoylaminooctadecane-1,3-diol,
2-N-[2-hydroxypalmitoyl]aminooctadecane-1,3-diol,
2-N-stearoylaminooctadecane-1,3,4-triol and in particular N-stearoylphytosphingosine,
2-N-palmitoylaminohexadecane-1,3-diol,
bis(N-hydroxyethyl-N-cetyl)malonamide,
N-(2-hydroxyethyl)-N-(3-cetyloxy-2-hydroxypropyl)cetylamide,
N-docosanoyl-N-methyl-D-glucamine,
or mixtures of these compounds.

34. The composition as claimed in any one of the preceding claims, characterized in that the protecting and/or conditioning agent(s) is (are) present in a concentration of between 0.001% and 20% by weight and preferably between 0.01% and 10% by weight relative to the total weight of the composition.

35. The composition as claimed in any one of the preceding claims, characterized in that it also comprises at least one surfactant chosen from anionic, nonionic and amphoteric surfactants, and mixtures thereof.

36. The composition as claimed in claim 35, characterized in that the surfactant(s) is (are) present in a concentration of between 0.1% and 60% by weight, preferably between 1% and 40% by weight and even more preferably between 5% and 30% by weight, relative to the total weight of the composition.

37. The composition as claimed in any one of the preceding claims, characterized in that it is in the form of a shampoo, a conditioner, a composition for permanent-waving, straightening, dyeing or bleaching the hair, a rinse-out composition to be applied between the two steps of a permanent-waving or hair-straightening operation, or a washing composition for the body.

38. The use of a composition as defined in any one of the preceding claims, for washing or caring for keratin materials.

39. A process for treating keratin materials, such as the hair, characterized in that it consists in applying to said materials a cosmetic composition as claimed in one of claims 1 to 37, optionally followed by rinsing with water.

40. The use of a cationic poly(alkyl)vinyllactam polymer as defined in one of claims 1 to 12 in or for the manufacture of a cosmetic composition comprising at least one protecting and/or conditioning agent.
